(19) United States
(12) Patent Application Publication
(10) Pub. No.: US 2004/0167207 A1
(43) Pub. Date: Aug. 26, 2004

(54) Combination therapy for anticoagulation

(76) Inventor: James M. Nesselroad III, Wataga, IL (US)

Correspondence Address: Allan L. Harms, Suite 420, 2750 First Avenue N.E., Cedar Rapids, IA, 52402, US

(21) Appl. No.: 10/752,788

(22) Filed: Jan. 7, 2004

Related U.S. Application Data
(60) Provisional application No. 60/439,090, filed on Jan. 8, 2003.

(51) Int. Cl. A61K 31/366; A61K 31/12

(52) U.S. Cl. 514/457; 514/682

(57) ABSTRACT

A combination anticoagulation medicament including vitamin K with warfarin in an oral form is described. Between 50 and 5000 micrograms of vitamin K are combined in a single oral medication with 0.5 to 15 milligrams of warfarin for administration. The combination of vitamin K with warfarin in a single orally dosed form is a novel approach to improving the effectiveness of anticoagulation. The combination allows for broader application of warfarin in medical anticoagulation and reduces the variability of anticoagulation due to the influences of diet, additional medications, nutritional status, changes in physical condition, and potentially other factors. Use of the combination therapy improves the safety of warfarin as an appropriate anticoagulant for many medical conditions.

### CROSS-REFERENCE TO RELATED APPLICATIONS

This application claims priority from copending provisional patent application entitled “Combination Therapy for Anticoagulation”, serial No. 60/439,090 filed Jan. 8, 2003, the disclosure of which is hereby incorporated in its entirety.

### FIELD OF THE INVENTION

The present invention relates generally to warfarin anticoagulation.

### BACKGROUND OF THE INVENTION

The medical use of warfarin as an oral anticoagulant is well known in the medical community. See, e.g. THE MERCK INDEX, AN ENCYCLOPEDIA OF CHEMICALS, DRUGS, AND BIOLOGICALS, (S. Budavari, Ed.), Twelfth Edition (1996). Warfarin is an effective therapy for many medical conditions, such as atrial fibrillation, myocardial infarction, artificial heart valves, venous thrombosis, and pulmonary embolism. Warfarin is also indicated for use as a medical anticoagulant in blood clotting disorders such as antiphospholipid syndrome. In these medical conditions, anticoagulation with warfarin reduces the risk of blood clot formation within the vasculature, which is termed thrombosis; and movement of such a blood clot through the vasculature, which is termed embolization.

The use of warfarin is limited by well-known side effects that can be disastrous for the patient. The most serious risks are hemorrhage in tissue or in an organ which may result in permanent disability or death. These risks are related to the level of intensity and the duration of warfarin treatment such as during anticoagulation therapy. The risk of serious hemorrhage may also be related to several patient specific conditions, including diet, age, history of gastrointestinal bleeding, history of stroke, anemia, hypertension, poor control of anticoagulation, excursions of anticoagulation level outside of the therapeutic range, usage of other drugs that impair other steps in the coagulation system, and thrombocytopenia. A patient's sensitivity to warfarin is also important, as the more sensitive a patient is to warfarin, the greater the risk for hemorrhagic complication.

Crystalline warfarin sodium is an anticoagulant which acts by inhibiting vitamin K-dependent coagulation factors. Chemically, crystalline warfarin sodium is 3-(_-acetonylbenzyl)-4-hydroxycoumarin and is a racemic mixture of the R and S enantiomers. Crystalline warfarin sodium is an isopropanol clathrate. The crystallization of warfarin sodium virtually eliminates trace impurities present in amorphous warfarin sodium. Crystalline warfarin sodium's empirical formula is C₁₉H₁₅NaO₄.

The formation of a clot is as a result of two hemostatic pathways: the primary and the secondary pathways. The primary pathway involves the formation of a platelet plug via platelet adhesion to the damaged subendothelium, granule release, and then platelet activation. The end result of this biochemical pathway is platelet aggregation (activated platelets sticking to each other) and the growth of the platelet plug. The secondary pathway involves the formation of fibrin. Clotting factors produced in the liver interact with each other to activate fibrinogen to an end product—fibrin monomer—which then polymerizes into an insoluble gel. Individual polymers/chains of fibrin are then cross-linked, which then stabilizes the platelet plug.

In order for clotting factors II, VII, IX and X to be active, they need to be carboxylated. This carboxylation is dependent on vitamin KH₂, which is the reduced form of vitamin K. Vitamin KH₂ is generated when vitamin K is reduced by vitamin K reductase. During carboxylation of the clotting factors, vitamin KH₂ is simultaneously oxidized to vitamin K epoxide (vitamin KO). Vitamin KO is in turn recycled to vitamin K by vitamin KO reductase. Warfarin mainly inhibits vitamin KO reductase; but warfarin also weakly inhibits vitamin K reductase. Although these two pathways are separate events, they are closely linked to each other. For example, during the formation of a clot, thrombin (factor IIa), induces platelet activation and conversely platelet activation accelerates the plasma coagulation via clotting factors.

Vitamin K is an essential cofactor for the synthesis of the clotting factors II, VII, IX, and X. Warfarin is an inhibitor of the inter-conversion of vitamin K and vitamin K epoxide, which causes the liver to produce coagulation factors with reduced effectiveness. Vitamin K and warfarin are competitive antagonists at the enzymatic level for synthesis of these clotting factors, with vitamin K promoting formation of active clotting factors and warfarin inhibiting formation.

Warfarin's anticoagulation effectiveness is influenced by anything affecting these biological pathways and chemical reactions, such as but not limited to other drugs, dietary vitamin K intake, changes in physical condition, and concurrent or acute medical illnesses. The anticoagulant response to warfarin is also influenced by drug interactions that affect its absorption and its clearance. For instance, many medications will reduce or increase the gastrointestinal absorption of warfarin. Other medications alter the plasma protein binding of warfarin and its serum concentration. Still other medications affect the metabolism of warfarin and reduce warfarin's clearance from the body. Many of these medically important interactions are listed in the Physician's Desk Reference. See, e.g., PHYSICIAN'S DESK REFERENCE, pg. 949 (49^{th} Ed., 1995).

The anticoagulant response to warfarin is also altered by variations in vitamin K intake, because vitamin K inhibits the anticoagulant action of warfarin. Because it is very difficult for an individual to maintain a consistent daily intake of vitamin K, variation in dietary vitamin K intake occurs daily. Eating a diet rich in vitamin K will ultimately require larger doses of warfarin for effective anticoagulation as the overabundance of vitamin K will shift the kinetics of the competing reactions to favor vitamin K over warfarin. However, in this situation, if the patient then varies from a diet containing vitamin K rich foods to a diet low in vitamin K, over-anticoagulation will occur as the previously required higher dosage of warfarin now produces a far higher percentage of defective anticoagulation factors than those effective anticoagulation factors produced by the reduced vitamin K. This situation risks the bleeding complications listed above as the person's blood may become dangerously overanticoagulated. The opposite situation occurs when a person who previously ate a low vitamin K diet and required a low dose of warfarin begins to consume a diet rich in vitamin K. Here, the low dosage of warfarin becomes outcompeted at the enzyme level by the higher intake of vitamin K. The coagulation factors are correctly produced in a far higher amount and this person's blood becomes underanticoagulated, with the corresponding risks of stroke or clot. These illustrations demonstrate the critical nature of diet in the anticoagulation patient, and the difficult but mandatory regulation of vitamin K intake. Therefore, current standard dietetic advice is to limit the intake of foods containing naturally high concentrations of vitamin K during treatment. In this fashion, the risk of variation of diet is reduced when vitamin K-containing foods are avoided entirely. And ultimately, the patient requires less warfarin for maintenance of anticoagulation. Yet, this dietary program, being the most common in use by warfarin anticoagulation patients, has a notable flaw that increases the risk of complications or adverse events. The reduced intake of vitamin K, combined with the reduced intake of warfarin, creates a more unstable situation and makes achieving appropriate anticoagulation more difficult, as discussed below.

Warfarin has a narrow therapeutic range, that is, the optimal dosing amount for medical patients is in a very small range. Since the development of the International Normalized Ratio, which is a method of reporting levels of anticoagulation consistently between different laboratories and testing techniques, physicians have been able to better focus warfarin anticoagulation therapy. Anticoagulation is monitored and the dosage of warfarin adjusted to maintain an International Normalized Ratio within a range specified by the condition which is being treated. For instance, atrial fibrillation is treated with warfarin to maintain an International Normalized Ratio between 2.0 and 3.0. Under a ratio of 2.0, the patient is insufficiently anticoagulated and at risk for thrombotic or embolic events. Over a ratio of 3.0, the patient is excessively anticoagulated. Increasing the level of anticoagulation does not further reduce the risk of thrombotic or embolic events, but it does increase the risk of hemorrhagic complications. Typical dosages can vary between about 2 and about 10 milligrams per day depending on the individual patient's physical condition and needs. Often, smaller than one milligram changes in the amount of warfarin taken daily will alter a patient's International Normalized Ratio beyond the range acceptable for the treated condition. Below the acceptable range for the International Normalized Ratio, patients do not derive the maximal benefit of anticoagulation from the warfarin medication. Above the acceptable International Normalized Ratio range, patients are at higher risk for developing hemorrhagic complications.

The amount of warfarin required to achieve effective anticoagulation also varies from patient to patient. For instance a large, youthful man may take ten milligrams of warfarin daily to maintain a clinically appropriate International Normalized Ratio, while a small, elderly man may require only two milligrams of warfarin daily to maintain the same clinically appropriate International Normalized Ratio. Further, due to many situations, some of which are described above, the range of medication will change in a particular patient over time. This means that a previously consistently appropriately anticoagulated patient may lose the effectiveness of their anticoagulation as their dosage requirement for warfarin changes in an unanticipated manner.

The amount of warfarin required to maintain an appropriate level of anticoagulation is also an important factor. In what would appear to be contrary to logic, patients who require a very small amount of warfarin to maintain an appropriate level of anticoagulation are at a higher risk for hemorrhagic complications. These patients are more likely to be elderly, chronically ill or taking additional medications that may alter their response to warfarin. In addition, any small change in the reaction dynamics, as previously discussed, will result in a much higher percent change in patients who have a low dosage of warfarin than those with a larger amount of warfarin in their systems. Any alteration in their fragile condition will adversely affect their anticoagulation level. In the example of the large, youthful man described above, his taking of ten milligrams of warfarin daily to maintain effective anticoagulation will likely result in a safer, more stable medical therapy than that of the small elderly man taking two milligrams of warfarin daily to maintain the same level of anticoagulation. While statistical analysis demonstrates the increased risk of complications with reduced doses of warfarin for effective anticoagulation, the pharmacological research has been unable to identify the cause of this phenomenon.

Even though physicians have an extensive amount of data concerning the effects of medical conditions, drugs, and diet on warfarin anticoagulation, the sensitivity of the situation is very difficult to maintain in a medically safe manner. And even with a vigilant patient's assistance, the anticoagulation effectiveness of a specific amount of warfarin may change, creating further difficulty in managing warfarin anticoagulation. It is to reduce these difficulties, and further assist the medical care of warfarin anticoagulation patients, that this invention is directed.

Therefore, there exists a need to provide an improved anticoagulation therapy and associated medication which reduces the variability of International Normalized Ratio levels in patients taking warfarin. In addition, there exists a need to reduce the impact of a patient's dietary intake of vitamin K-rich foods and of other medications, as well as variations in the state of health of the patient, on the effectiveness of the warfarin anticoagulation therapy. Still further, there exists a need to provide an improved medication for anticoagulation which facilitates maintenance of a medically appropriate International Normalized Ratio for a patient in need of warfarin anticoagulation. A need exists to provide a combination medication which reduces the risk of excessive anticoagulation or ineffective anticoagulation therapy.

### SUMMARY OF THE INVENTION

A medicament in accordance with the principles of the present invention provides an improved anticoagulation therapy and associated medication which reduces the variability of International Normalized Ratio levels in patients taking warfarin. A medicament in accordance with the principles of the present invention reduces the impact of a patient's dietary intake of vitamin K-rich foods and of other medications, as well as variations in the state of health of the patient, on the effectiveness of the warfarin anticoagulation therapy. A medicament in accordance with the principles of the present invention provides an improved medication for anticoagulation which facilitates maintenance of a medically appropriate International Normalized Ratio for a patient in need of warfarin anticoagulation. A medicament in accordance with the principles of the present invention provides a combination medication which reduces the risk of excessive anticoagulation or ineffective anticoagulation therapy.

A medicament in accordance with the principles of the present invention provides a combination medication for anticoagulation which includes therapeutic quantities of warfarin combined with substantial dosages of vitamin K. A fixed amount of vitamin K, for example between about 50 and about 250 micrograms (μg), is combined with warfarin, for example about 0.5 milligrams to about 15 mg, in for example an oral dosage form, namely a capsule or tablet. The combination oral medication is administered to a patient requiring anticoagulation therapy.

A method for treating patients who would benefit from anticoagulation includes administering vitamin K contemporaneously with warfarin to provide a predictable vitamin K serum level which is little impacted by varying quantities of dietary vitamin K, by the intake of other medications or by the medical condition of the patient. The maintenance of a predictable vitamin K serum level provides a predictable antagonist for warfarin causing reduction in the variability of International Normalized Ratio for the patient.

These and other objects of the invention will become apparent from examination of the description and claims which follow.

### DESCRIPTION OF THE FIGURES

FIG. 1 depicts the chemical structure of an example warfarin in accordance with the principles of the present invention.

### DETAILED DESCRIPTION OF THE PREFERRED EMBODIMENTS

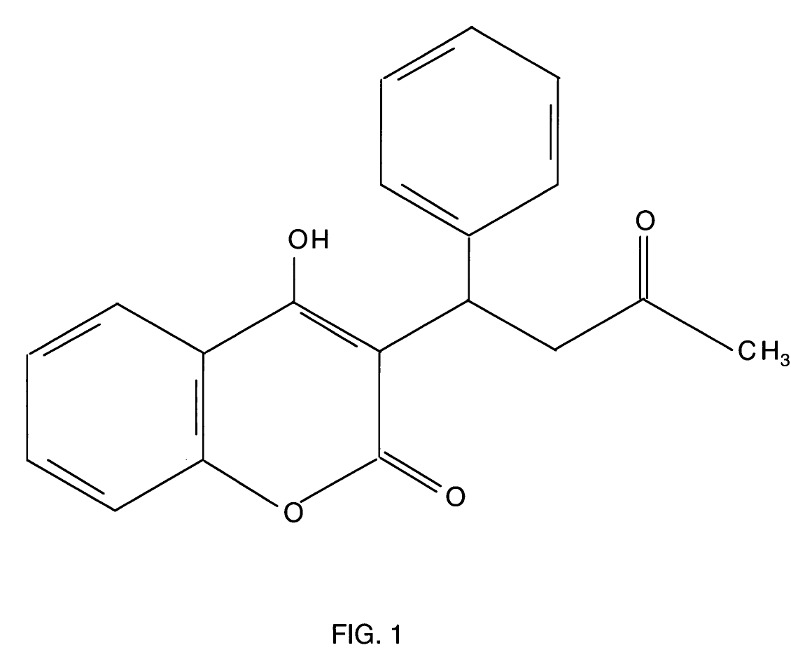

A medicament in accordance with the principles of the present invention provides an improved therapeutic anticoagulant medication by combining warfarin and vitamin K in an orally dosed form. Throughout this disclosure, the term “warfarin” shall include all medically active forms of warfarin including but not limited to warfarin sodium and all medically active salts of warfarin such as for example the compound illustrated in FIG. 1. Reference to vitamin K includes vitamin K1 and phytonadione.

In accordance with one embodiment of the present invention, combination of warfarin and vitamin K is accomplished by adding fixed amounts of each medication into an orally dosed form. The standard oral dosages of warfarin are (in milligrams): 0.5, 1, 1.25, 1.5, 1.75, 2.0, 2.25, 2.5, 2.75, 3.0, 3.25, 3.5, 3.75, 4.0, 4.5, 5.0, 5.5, 6.0, 6.5, 7.0, 7.5, 8.0, 8.5, 9.0, 9.5, 10, 11, 12, 13, and 15. The desired dosage of warfarin may be mixed with between about 50 and about 5000 micrograms of vitamin K, but preferably the vitamin K is provided in a range of about 100 to about 1000 micrograms; more preferably, the vitamin K is provided in a range of from about 100 micrograms to about 250 micrograms per day.

An orally dosed form in accordance with the present invention may take the form of a tablet or a capsule and may include pharmaceutically appropriate inert ingredients, excipients, and carrier materials appropriate to mass production of a medically useful orally dosed medication. Such inert ingredients may include but are not limited to lactose, starch, sucrose, glucose, modified sugars, modified starches, methyl cellulose, methyl cellulose derivatives, dicalcium phosphate, calcium sulfate, mannitol, sorbitol, magnesium stearate, stearic acid, sodium stearyl fumarate, glycerol behenate, gelatin, calcium stearate, waxes, synthetic gums and other necessary or suitable binders, coloring agents, and stabilizing agents.

As previously described, vitamin K is an essential cofactor for the synthesis of the clotting factors II, VII, IX, and X. Warfarin is an inhibitor of the interconversion of vitamin K and vitamin K epoxide, which causes the liver to produce coagulation factors with reduced effectiveness. Vitamin K and warfarin are competitive antagonists at the enzymatic level for synthesis of these clotting factors with vitamin K promoting formation of active clotting factors and warfarin inhibiting formation. The admixture of both entities into a single orally dosed form at specific ratios, reduces the variations in level of anticoagulation that can occur secondary to dietary variation, drug changes in the patient's medical care, changes in the patient's physical condition, acute medical illnesses, and potentially other factors. This reduction in variation of the level of anticoagulation makes the combination of warfarin and vitamin K a safer and more effective product for medical use in patients for whom warfarin anticoagulation is indicated. By adding vitamin K to warfarin, dietary variations are mitigated by the amount of vitamin K within the combination dosing.

The average individual consumes between about 80 and about 250 micrograms of vitamin K in their daily diet. This translates to a variance in excess of 300 percent in the amount of daily vitamin K intake. Since the warfarin dose needs to be adjusted to the dietary vitamin K intake, it has been established practice to recommend reducing the overall dietary vitamin K intake: but this method then reduces the amount of warfarin required for effective anticoagulation. Reduced doses of warfarin are more difficult to maintain and lead to further risk of inadequate anticoagulation or over anticoagulation.

By adding a fixed amount of vitamin K to the warfarin in a single daily dose, the dietary variation is significantly reduced. For instance, adding 500 micrograms of vitamin K to the daily dose, combined with the usual 80 to 250 micrograms of vitamin K in the diet, will result in a consumption of 580 to 750 micrograms per day of vitamin K intake (a variation of thirty percent (30%)). This reduction in variation makes the combination of vitamin K and warfarin in a daily dosed form a much safer and more effective product for medical use in patients for whom warfarin anticoagulation is indicated.

By adding vitamin K to warfarin, drug induced variations of warfarin effectiveness are mitigated in the following manner: The vitamin K contained within the combined dose requires a larger amount of warfarin for effective anticoagulation. A higher amount of warfarin within the dose results in a higher concentration of warfarin in the body. The higher concentration of warfarin in the body is more resistant to variations in level due to the influence of other drugs, in a similar manner as variance in the concentration of vitamin K is reduced when dosed vitamin K and dietary vitamin K are combined. The higher concentration of warfarin reduces the variations of warfarin concentration and achieves a more consistent effect. A higher level of warfarin alone, though more consistent, would dangerously anticoagulate the patient, creating its own dangerous hemorrhagic complications.

The addition of vitamin K to warfarin in accordance with the principles of the present invention makes possible the use of larger doses of each, thereby enhancing safety from the reduction in dietary vitamin K variation and also from the reduction in drug induced variations in warfarin concentration. Standard medical therapy to this point has been to minimize the amounts of each, creating inherent instability due to the very small levels of each in the serum. The present invention counter-intuitively uses larger doses of each, and by combining vitamin K and warfarin in a single form, creates greater stability against these variations in the serum, and greater safety. This is a more effective product for medical use in patients for whom warfarin anticoagulation is indicated.

The invention may also be practiced by prescribing the concurrent intake of about 50 to about 5000 micrograms, preferably about 100 to about 1000 micrograms, and more preferably from about 100 to about 250 micrograms per day, of vitamin K, along with an oral dosage of warfarin, while monitoring the International Normalized Ratio level of the patient to lead to adjustment of the warfarin intake to achieve therapeutic and safe anticoagulation. Therefore the patient may be directed to ingest daily this combination of vitamin K tablet in the range of about 100 to about 1000 micrograms and warfarin in the range of about 0.5 to about 15 micrograms per day in order to maintain a medically safe and more consistent level of anticoagulation than can be achieved by warfarin alone.

### EXAMPLE

The following is a non-limiting example of the administration of a medicament in accordance with the present invention:

In a study conducted to demonstrate the effectiveness of the present invention, 24 patients consented to participate and were randomized into three study groups: a control group receiving warfarin alone; a study group receiving combination warfarin and vitamin K in a single oral preparation; and a second experimental group receiving warfarin dosing via a standardized algorithm depicted in Tables 1 and 2.

TABLE 1
INR Therapy for Established Patents Who are Indicated for INR of 2.0-3.0

| | Last Check was done . . . | | |
|---|---|---|---|
| INR | 3 days ago* | 4 days ago* | 1 week ago |
| Under 1.50 | Increase dose 2 levels. | Increase dose 2 levels. | Confirm patient is taking Coumadin dose |
| | Recheck in 3 days. | Recheck in 3 days. | daily. |
| | | | If yes increase the does 2 levels and recheck in |
| | | | 3 days. |
| | | | If no, confirm daily dose and recheck in 3 days. |
| 1.50-1.74 | Increase does 1 level. | Increase dose 1 level. | Increase dose 1 level. |
| | Recheck in 3 days. | Recheck in 3 days. | Recheck in 4 days. |
| 1.75-1.99 | Increase dose 1 level. | Increase dose 1 level. | Increase dose 1 level. |
| | Recheck in 3 days. | Recheck in 4 days. | Recheck in 3 days. |
| 2.00-3.00 | No dose change. | No dose change. | No dose change. |
| | Recheck in 4 days. | Recheck in 1 week. | Recheck in 1 week. |
| 3.01-3.25 | Decrease dose 1 level. | Decrease dose 1 level. | Decrease dose 1 level. |
| | Recheck in 3 days. | Recheck in 4 days. | Recheck in 4 days. |
| 3.26-3.50 | Decrease dose 1 level. | Decrease dose 1 level. | Decrease dose 1 level. |
| | Recheck in 3 days. | Recheck in 3 days. | Recheck in 3 days. |
| 3.51-4.00 | Decrease dose 2 levels. | Decrease dose 2 levels. | Decrease dose 2 levels. |
| | Recheck in 3 days. | Recheck in 3 days. | Recheck in 3 days. |
| 4.01-4.50 | Decrease dose 2 levels. | Decrease dose 2 levels. | Decrease dose 2 levels. |
| | Recheck in 3 days. | Recheck in 3 days. | Recheck in 3 days. |
| Over 4.50 | Add vitamin K 5 mg. | Add vitamin K 5 mg. | Add vitamin K 5 mg. po. |
| | po. | po. | Decrease dose 2 levels. |
| | Decrease dose 2 levels. | Decrease dose 2 levels. | Recheck in 3 days. |
| | Recheck in 3 days. | Recheck in 3 days. | |

TABLE 2
Dosage configurations.

| Total mg | Dose |
|---|---|
| 0.5 | ½ of 1 mg |
| 1.0 | 1 mg |
| 1.25 | ½ of 2.5 mg |
| 1.5 | ½ of 3 mg |
| 1.75 | ½ of 2.5 mg + ½ of 1 mg |
| 2.0 | 2 mg |
| 2.25 | ½ of 2.5 mg + 1 mg |
| 2.5 | 2.5 mg |
| 2.75 | ½ of 2.5 mg + ½ of 3 mg |
| 3.0 | 3 mg |
| 3.25 | ½ of 2.5 mg + 2 mg |
| 3.5 | 2.5 mg + 1 mg |
| 3.75 | ½ of 7.5 mg |
| 4.0 | 4.0 mg |
| 4.5 | 2 mg + 2.5 mg |
| 5.0 | 5 mg |
| 5.5 | 5 mg + ½ of 1 mg |
| 6.0 | 5 mg + 1 mg |
| 6.5 | 4 mg + 2 _{—} mg |
| 7.0 | 5 mg + 2 mg |
| 7.5 | 7.5 mg |
| 8 | 5 mg + 3 mg |
| 8.5 | 7.5 mg + 1 mg |
| 9 | 5 mg + 4 mg |
| 9.5 | 7.5 mg + 2 mg |
| 10 | 10 mg |
| 11 | 10 mg + 1 mg |
| 12 | 10 mg + 2 mg |
| 13 | 10 mg + 3 mg |

The algorithm group was found to not be statistically significant from the control group. But the combination warfarin—vitamin K of the present invention was shown to be a statistically significant improvement over standard medical therapy with warfarin alone. Over six months of study, in which these 24 patients had their anticoagulation level checked by International Normalized Ratios (INRs) on at least a weekly basis, the following important facts were established.

The number of clinically safe anticoagulation levels (defined as INR level between 2.0 and 3.0) was higher in the combination warfarin—vitamin K group when compared to the control group [19.83 versus 14.83, t(22)=−2.283, p=0.032]. The data thus supports the fact that the combination provided more appropriate and clinically accurate anticoagulation.

The number of medically safe anticoagulation levels (defined as INR level between 1.8 and 3.5) was higher in the combination warfarin—vitamin K group [23.92 versus 18.42, t(22)=−2.09, p=0.048].

The number of medically unsafe INRs (defined as INR less than 1.5 or greater than 4.9) was numerically lower in the warfarin—vitamin K group, but did not meet statistical significance due to the low number of study participants [27.83 versus 22.00, t(22)=−2.032, p=0.054].

The number of anticoagulation dose changes in response to changes in concomitant medication regimens was lower in the warfarin—vitamin K group than in the control group. Four instances required anticoagulation adjustment after other drug therapy changes in the warfarin—vitamin K group, while 10 instances occurred in the control group. This indicates that the combination did in fact help patients resist changes in anticoagulation due to adjustments in concomitant drug therapy.

### Evaluation Study

Adult anticoagulation patients presenting to the practice site were informed of the study, and the opportunity to be a test subject. Subjects were required to complete a study entry data interview, provide informed consent, and present to the clinic once to twice weekly for INR evaluation. The control group and a second group (the algorithm group) of patients were provided warfarin alone as an anticoagulation agent, while the study group patients received warfarin and 100 μg of vitamin K in a single gelatin capsule from a single pharmacy. This pharmacy also tracked refill data and provided statements confirming patient compliance throughout the study. The study received clearance from the St. Mary's Medical Center Institutional Review Board, Galesburg, Ill.

An initial study interview was conducted and identified a patient's sex, age, indication for anticoagulation, and prescription and nonprescription medication usage. Those who were appropriate for study and provided consent were required to present to the clinic for once or twice weekly INR testing.

The control group (N=6) received their warfarin dosed by conventional tablet forms and presented to the office at least weekly for INR labs (more often if dosage changes were required).

The algorithm group (N=6) received their warfarin based upon an algorithm devised by the investigators to standardize the adjustments and the dosing of warfarin in smaller increments than those available in conventional dosages of oral medication (tablets). The algorithm specified dosages of warfarin other than those used by the manufacturer, in order for the prescribing physician to specify smaller incremental dosage changes than those permitted with the standard dosages available.

The purpose of having the algorithm group as a part of the study was to determine if the algorithm played a significant role in reducing nontherapeutic anticoagulation intervals. The first theory behind the algorithm is that as patients remain stably anticoagulated, their INR frequency would be gradually reduced to weekly visits. If a dosing adjustment was required due to a nontherapeutic INR, the dose of warfarin would be adjusted in a fixed increment and the time of the next INR set by reducing the interval.

The second theory behind the algorithm is that at the lower warfarin doses, the interval between easily taken amounts is a significant dose change. For example, the increase in dosage between 1 mg and 2 mg daily is a 100 percent dose increase. In comparison, the increase between the 4 mg tab and the 5 mg tab is a 25 percent dose increase. In order to create smaller dosage steps between doses of warfarin, the various available tablets were halved along their scoring lines and the appropriate amount was placed into a gelatin capsule for easy daily dosing. For instance, a dose of 1.75 mg warfarin required one-half of a 2.5 mg tablet and one-half of a 1 mg tab to be placed into a gelatin capsule. A cooperating compounding pharmacist placed the daily doses of warfarin into gelatin capsules for the use of the patients in the algorithm group.

The study group (N=12) received warfarin combined with 100 μg of vitamin K compounded into a gelatin capsule by the compounding pharmacist. The warfarin was dosed by the study algorithm, described above. Patients who randomized to this group received their current dose of warfarin along with the dose of vitamin K in the daily gelatin capsule. And as the INR fluctuated, the dose of warfarin was adjusted by the algorithm.

The combination of warfarin and vitamin K was accomplished by adding fixed amounts of each into an orally dosed form. The doses of warfarin used within the study protocol were (in milligrams): 0.5, 1, 1.25, 1.5, 1.75, 2.0, 2.25, 2.5, 2.75, 3.0, 3.25, 3.5, 3.75, 4.0, 4.5, 5.0, 5.5, 6.0, 6.5, 7.0, 7.5, 8.0, 8.5, 9.0, 9.5, 10, 11, 12, 13, 15. The selected dose of warfarin was combined in the gelatin capsule with 100 micrograms of vitamin K.

### Results of the Study

The theory behind this study is the probability that adding vitamin K in a standardized daily dose would reduce the variations in anticoagulation effectiveness of warfarin due to external factors. The admixture of both entities into a single orally dosed form at specific ratios, reduces the variations in level of anticoagulation that can occur secondary to dietary variation, drug changes in the patient's medical care, changes in the patient's physical condition, acute medical illnesses, and potentially other factors. This reduction in variation of level of anticoagulation makes the combination of warfarin and vitamin K a safer, more effective product for medical use in patients for whom warfarin anticoagulation is indicated.

Twenty-four subjects completed the study (14 females and 10 males, mean age 68.9 years, age range from 43 to 88 years). Three subjects underwent surgery during the study, and as a result were medically required to discontinue their anticoagulation. After these patients recuperated sufficiently to resume anticoagulation, their data collection was resumed. Up to 35 INR measurements were taken by these patients over the six months.

The control group and the algorithm group did not have a statistically meaningful difference in their warfarin doses or INR results. Therefore, the algorithm patients were not at any risk when their warfarin dose was adjusted based on the algorithm as opposed to physician dose adjustment. Also, in order to create a base of data for the control versus the addition of vitamin K, these two groups were combined. This made analysis more statistically important, as with this study, small numbers make for more difficult interpretation.

Outcome measures included the number of clinically safe INRs (INR range=2.0 to 3.0); number of medically safe INRs (INR range 1.8 to 3.5; and INR range 1.5 to 4.9); number of INR levels suggesting under-anticoagulation (INR less than 1.5); INR level suggesting over-anticoagulation (INR greater than 5.0); and anticoagulation dose changes after a change in concomitant medication. The data was entered into a computerized database with Microsoft Excel spreadsheet program. Data analysis was conducted using SPSS 11.5 statistical analysis software, which is available from SPSS Inc. 233 S. Wacker Drive 11th Floor Chicago, Ill. 60606.

The use of the combination preparation containing warfarin and vitamin K resulted in more consistent warfarin dosing in the experimental group receiving the combination capsule. Across the first 24 measures of this study, there was only a 15% variation from low to high doses (4.25-5.00 mgs) in this group. By contrast, the average dose for the algorithm only arm demonstrated a 25.3% variation (4.71-6.30 mgs). Further, the patients in the standard care group demonstrated a 36.9% variation in dosing from low to high dose (3.50-5.55 mgs) This indicates that the addition of vitamin K improved the stability of anticoagulation in these patients. It also reduced the variation in amount of warfarin required to maintain appropriate anticoagulation.

The use of the vitamin K—warfarin combination resulted in patients having a larger number of clinically therapeutic INR's (2.0-3.0). The data showed more INR values in the therapeutic range in patients receiving the combination therapy [19.83 versus 14.83, t(22)=−2.283, p=0.032].

The use of vitamin K—warfarin combination resulted in a larger number of medically safe INRs (INR range 1.8-3.5) [23.92 versus 18.42, t(22) =-2.09, p=0.048].

The use of vitamin K—warfarin combination led to numerically fewer INR levels outside of the medically safe range (1.5-4.9); however this did not achieve a criterion level of significance [27.83 versus 22.00, t(22)=-2.032, p=0.054].

The use of the combination vitamin K—warfarin capsule reduced the number of anticoagulation dosage adjustments required after a change in concomitant medical therapy. Four patients in the combination group required an adjustment in their anticoagulant dose after a change in medication; however, ten instances were found in the control and the algorithm arms of the trial. Eight persons (including the two subjects who had this occur twice) in these groups had a dosage change in their anticoagulation after a change in concomitant medical therapy.

The foregoing description of the invention has been presented for purposes of illustration and description and is not intended to be exhaustive or to limit the invention to the precise form disclosed. Modifications and variations of the embodiments are possible in light of the above disclosure or such may be acquired through practice of the invention. The embodiments illustrated were chosen in order to explain the principles of the invention and its practical application to enable one skilled in the art to utilize the invention in various embodiments and with various modifications as are suited to the particular use contemplated. It is intended that the scope of the invention be defined by the claims appended hereto, and by their equivalents.

What is claimed is:

1. A therapeutic preparation comprising:
warfarin; and
vitamin K.

2. The therapeutic preparation of claim 1 wherein the quantity of vitamin K is in the range of about 50 to about 5000 micrograms.

3. The therapeutic preparation of claim 1 wherein the quantity of vitamin K is in the range of about 100 to about 1000 micrograms.

4. The therapeutic preparation of claim 1 wherein about 0.5 to about 15 milligrams of warfarin is combined with from about 50 to about 5000 micrograms of vitamin K.

5. The therapeutic preparation of claim 1 wherein about 0.5 to about 15 milligrams of warfarin is combined with from about 100 to about 1000 micrograms of vitamin K.

6. The therapeutic preparation of claim 1 wherein about 0.5 to about 15 milligrams of warfarin is combined with from about 100 to about 250 micrograms of vitamin K.

7. The therapeutic preparation of claim 1 wherein vitamin K is combined with warfarin.

8. The therapeutic preparation of claim 1 wherein phytonadione is combined with warfarin.

9. The therapeutic preparation of claim 1 wherein the warfarin and vitamin K are combined in an oral medication.

10. An oral medication comprising: warfarin and vitamin K.

11. The oral medication of claim 10 wherein the quantity of vitamin K is in the range of about 50 to about 5000 micrograms.

12. The oral medication of claim 10 wherein the quantity of vitamin K is in the range of about 100 to about 1000 micrograms.

13. The oral medication of claim 10 wherein about 0.5 to about 15 milligrams of warfarin is combined with from about 50 to about 5000 micrograms of vitamin K.

14. A method of treating persons needing anticoagulation therapy comprising the step of:
administering warfarin concurrently with the administration of vitamin K.

15. The method of claim 14 wherein from about 50 to about 5000 micrograms of Vitamin K is administered with warfarin.

16. The method of claim 14 wherein from about 100 to about 1000 micrograms of Vitamin K is administered with warfarin.

17. The method of claim 14 wherein from about 0.5 to about 15 milligrams of warfarin is administered with from about 50 to about 5000 micrograms of vitamin K.

18. The method of claim 14 wherein from about 0.5 to about 15 milligrams of warfarin is administered with from about 100 to about 1000 micrograms of vitamin K.

19. The method of claim 14 wherein the warfarin and the vitamin K are combined in an oral dosage.

20. A composition comprising:
a medically effective dosage of a chemical having the formula:

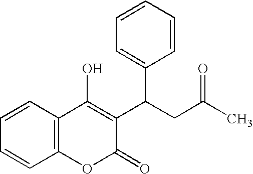

21. The composition of claim 20 wherein the quantity of vitamin K is in the range of about 50 to about 5000 micrograms.

22. The composition of claim 20 wherein the quantity of vitamin K is in the range of about 100 to about 1000 micrograms.

23. The composition of claim 20 wherein about 0.5 to about 15 milligrams of warfarin is combined with from about 50 to about 5000 micrograms of vitamin K.

24. The composition of claim 20 wherein about 0.5 to about 15 milligrams of warfarin is combined with from about 100 to about 1000 micrograms of vitamin K.

25. The composition of claim 20 wherein about 0.5 to about 15 milligrams of warfarin is combined with from about 100 to about 250 micrograms of vitamin K.

26. The composition of claim 20 further comprising excipients.

27. The composition of claim 20 wherein phytonadione is combined with warfarin.

28. The composition of claim 20 wherein the warfarin and vitamin K are combined in an oral medication.

29. The composition of claim 28 wherein the oral medication is a capsule.

30. The composition of claim 28 wherein the oral medication is a tablet.
